(19) United States
(12) Patent Application Publication
(10) Pub. No.: US 2006/0095013 A1
(43) Pub. Date: May 4, 2006

(54) Changing cap

(76) Inventor: Denise Hayman, Pacific Palisades, CA (US)

Correspondence Address: CHRISTIE, PARKER & HALE, LLP, PO BOX 7068, PASADENA, CA, 91109-7068, US

(21) Appl. No.: 10/982,289

(22) Filed: Nov. 4, 2004

(51) Int. Cl. A61F 13/15

(52) U.S. Cl. 604/385.09

(57) ABSTRACT

Changing apparatus for preventing spraying incidence when changing a child's diaper with the child's private part exposed is generally discussed herein with particular discussions extended to changing caps for capping the child's private part when changing the child's diaper. The changing caps may have different mechanisms for latching onto the child, different designs to appeal to the child, and may include an insert for absorbing moisture excreted by the child. The changing cap may also include moisture detection mechanism for detecting wetness and may be used for other applications.

Changing apparatus for preventing spraying incidence when changing a child's diaper with the child's private part exposed is generally discussed herein with particular discussions extended to changing caps for capping the child's private part.

### BACKGROUND

Changing a child's diaper when he or she soils it can take several minutes. During this procedure, the parent will generally need to remove the soiled diaper and clean the child with tissues and/or wet wipes. In some instances, the child is then rinsed with soap and water. If the child merely pees, the parent will sometime take a cloth or a towel to cover the child's private part while cleaning and changing the child's diaper. The cloth or towel provides a barrier and prevents spraying in the event the child should pee while the parent changes his or her diaper. In addition, the towel allows the parent to take his or her time to thoroughly clean the child without the fear of getting sprayed by an exposed child.

However, if the child has a bowel movement, using a cloth or a towel to prevent spraying may not be wise because the towel can come in contact with the fecal matter. In this situation, the parent may forego using the towel and opt instead for the odds of not getting sprayed, i.e., take his or her chances. Ideally, the parent should take the necessary time to thoroughly clean the child for obvious hygiene reasons. Unfortunately, the chance of getting sprayed also increases when the child's private part is left exposed for a longer period of time, especially when changing the child following a bowel movement.

Accordingly, there is a need for a changing cap that enables the parent to cover the child's private part during a diaper change or during other activities.

### SUMMARY

The present invention may be practiced using the method of covering a child comprising removing a diaper; and placing a cap comprising an exterior surface, an interior surface defining an interior cavity, and an opening having a largest measured opening dimension of between 1.5 inches to 4 inches over a urinary system of the child without covering a buttock of the child.

The present invention may also be practiced by providing an apparatus for covering a child comprising a cap comprising an interior cavity and an elastic stretch band adjacent a perimeter defining an opening; an absorbent insert positioned inside the interior cavity of the cap; and wherein the opening is sized to fit around a genital area of the child.

The present invention may yet be practiced by providing an apparatus for covering a child comprising a cap comprising an exterior surface having an exterior color, an interior surface defining an interior cavity, and an elastic stretch band adjacent a perimeter defining an opening; a decorative element adorns the exterior surface of the cap to provide a distinct visual indicia apart from the exterior color; and wherein the opening is sized to contact a genital area of the child around a substantial portion of the opening.

### BRIEF DESCRIPTION OF THE DRAWINGS

These and other features and advantages of the present invention will become appreciated as the same become better understood with reference to the specification, claims and appended drawings wherein:

FIG. 1 is a semi-schematic perspective view of a changing cap for capping the private part of the child after removing the child's diaper;

FIG. 2 is a semi-schematic perspective view of the changing cap of FIG. 1 capping the private part of a child;

FIG. 3 is a semi-schematic drawing of an alternative changing cap provided in accordance with aspects of the present invention;

FIG. 4 is a semi-schematic perspective view of the changing cap of FIG. 3;

FIG. 5 is a semi-schematic top view of an alternative changing cap provided in accordance with aspects of the present invention;

FIG. 6 is a semi-schematic plan view of an exemplary insert usable with the changing caps of the present invention.

### DETAILED DESCRIPTION

The detailed description set forth below in connection with the appended drawings is intended as a description of the presently preferred embodiments of changing caps provided in accordance with aspects of the present invention and is not intended to represent the only forms in which the present invention may be constructed or utilized. The description sets forth the features and the steps for constructing and using the changing caps of the present invention in connection with the illustrated embodiments. It is to be understood, however, that the same or equivalent functions and structures may be accomplished by different embodiments that are also intended to be encompassed within the spirit and scope of the invention. Also, as denoted elsewhere herein, like element numbers are intended to indicate like or similar elements or features.

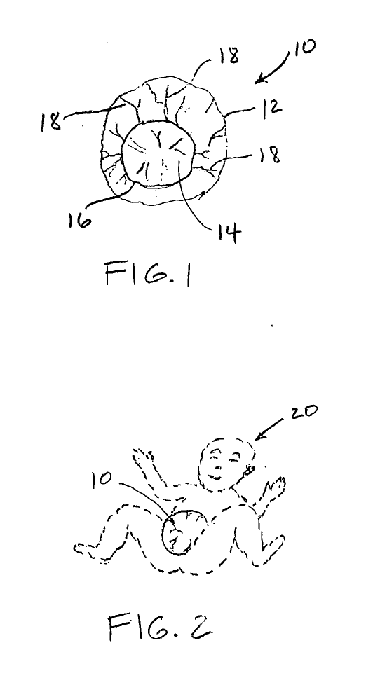

Referring now to FIG. 1, there is shown a semi-schematic drawing of a changing cap provided in accordance with aspects of the present invention, which is generally designated 10. In one exemplary embodiment, the changing cap 10 resembles a shower cap with at least the following differences: it is smaller in size relative to a standard shower cap and it may include an absorbent material for reasons further discussed below. In one exemplary embodiment, the cap 10 is made from a washable polyurethane laminated polyester material. However, other plastic and plastic blend materials, such as vinyl and polyethylene, may be used to make the changing cap 10 without deviating from the spirit and scope of the present invention. The plastic or plastic blend material allows the cap to be washed for reuse. In yet another alternative embodiment, the changing may be disposable after or more use or at least until it gets dirty or contaminated.

In one exemplary embodiment, the changing cap comprises a cover or a housing 12 comprising an opening 14 having an elastic stretch band 16 forming the circumference of the opening defining the opening. The cap 10 may be formed from a generally circular sheet of plastic or plastic blend material, folding the sheet into a ball-like configuration while attaching the elastic stretch band 16 around the perimeter of the sheet to form the opening 14. Because the elastic stretch band 16 collapses or pulls in the perimeter of the sheet to form a ball-like configuration a plurality of creases 18 are formed. The foregoing described steps for making the changing cap 10 are well known in the field of shower caps.

In an exemplary embodiment, the generally circular plastic or plastic blend sheet used to form the cover or housing 12 is about 4 to about 16 inches in diameter with about 6 to about 12 inches being more preferred. The opening 14, which is generally circular and takes on the shape of the perimeter of the sheet, is about 1.5 inches to about 4 inches in diameter, when in the unstretched position, i.e., before placing on the child. In general, the opening should have a largest measured opening dimension of between about 1.5 inches to about 4 inches in diameter

Referring now to FIG. 2, a semi-schematic drawing of the cap 10 placed on a child 20 is shown. In particular, the cap 10 is placed on the child's private part. Although the changing cap 10 may be used on a female child, it is better adapted for use on a male child. The cap 10 may be used by placing the opening over the child's penis, which is sometimes referred to as the genital area or private part, and ensuring that the elastic stretch band 16 is tucked under the child's testicles. To ensure that the cap 10 is sufficiently secured so that it does not accidentally fall off during the diaper changing procedure, the elastic stretch band 16 should be sized so that it stretches sufficiently and the elasticity of the stretch band creates a small gripping force around the child's private part. Once the cap 10 is mounted over the child 20, the risk of being sprayed by the child is eliminated. Thus, the parent can now take his or her time to change the child's diaper, leave the child exposed to air out the child's bottom for a prolonged period, such as 5 minutes or more, or for any other activities, such as while waiting for or during a doctor's visit, to weigh the child, to take naked childhood pictures of the child, etc.

Referring now to FIG. 3, a semi-schematic drawing of an alternative changing cap 22 is shown in an un-folded configuration for clarity. The alternative cap 22 may be constructed from a sheet 24 of plastic or plastic blend material, creating a cut-out 26 on the sheet, reinforcing the cut-out by stitching backing material or bonding backing material 28 along the cut-out, adding a length of elastic stretch band 16 around a portion of the perimeter of the sheet 24, and adding an adjustable connector 30 adjacent the cut-out 26. Exemplary adjustable connectors include buttons and button holes, Velcro®, and snap-buttons. Exemplary reinforcing alternatives for the cut-out 26 include stitching the cut-out with an overlock stitch, and overlapping the cut-out on itself and stitching the overlapped sections. Other conventional reinforcing methods are contemplated and are considered to fall within the spirit and scope of the present invention.

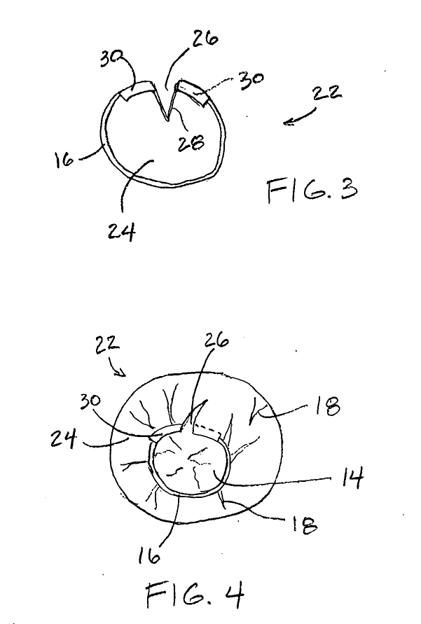

Referring now to FIG. 4, a semi-schematic drawing of the changing cap 22 of FIG. 3 is shown in its normal ball-like configuration. As is readily apparent, the cut-out 26 and the adjustable connector 30 allows the opening 14 of the cap 22 to be adjusted when mounted on the child's private part to provide a secured attachment. The opening 14 may be adjusted by varying the placement of a button relative to a number of button holes, varying the placement of a male button snap relative to a number of female button snaps, or varying the placement of a loop versus a hook if Velcro® is used. Caution should be taken to ensure against over tightening around the child's private part for obvious safety reasons.

In an alternative embodiment, two or more cut-outs along with two or more adjustable connectors may be used. Still yet in another alternative embodiment, the shape of the cap (in FIGS. 1 and 4) may be changed from a ball-like configuration to irregular shapes, such as an oval shape, a football shape, a baseball bat shape, or an animal shape (i.e., elephant, pig, horse, etc.). Still alternatively, the cover or housing may be decorated with designs or visual indicia comprising different colors, bright colors, embossed, artistic prints, etc. Other visual indicia or configurations include a fireman's had, a policeman's hat, a motorcycle helmet, and a bicycle helmet.

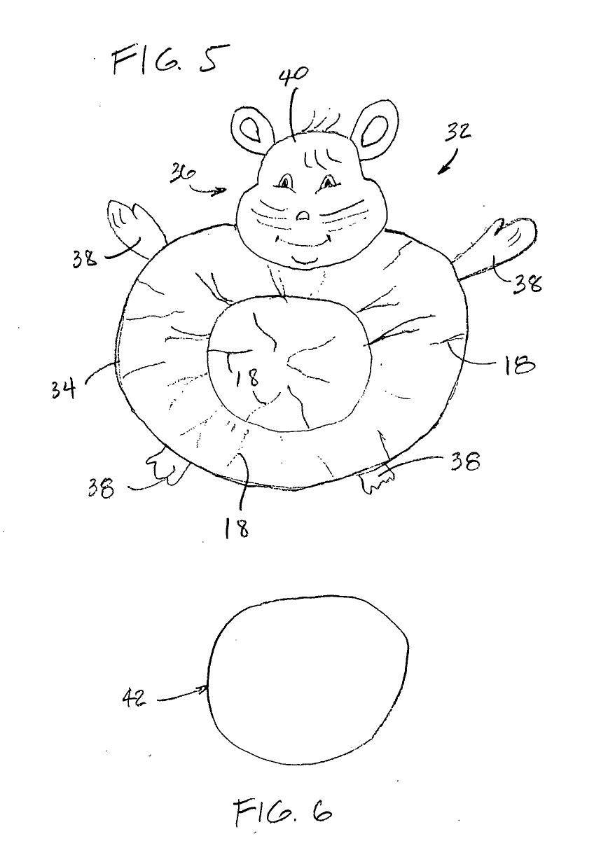

Referring now to FIG. 5, an alternative changing cap 32 provided in accordance with aspects of the present invention is shown. The alternative changing cap 32 is shown comprising a cover or housing 34 that is similar to the cover 12 of FIG. 1, which may optionally be similar to the cover 22 of FIG. 4. In one exemplary embodiment, the cap 32 comprises an artistic or design indicia 36 for altering the appearance of the cap 32. In one exemplary embodiment, the artistic indicia 36 comprises features of a fish or an animal, such as a cat, a dog, an elephant, a whale, etc. As shown, the artistic indicia 36 comprises a set of paws 38 and the head of a rat 40. The artistic indicia can comprise printed plastic figurines or stuffed objects sewn onto or heat bonded to the cap.

FIG. 6 is a semi-schematic view of a moisture absorbing insert 42 shown in a generally circular configuration. However, the insert 42 may be made from other shapes including an oval shape, a star shape, a rectangular shape, and a square shape. The insert 42 may be a reusable type, which is washable, or a disposable type. Exemplary reusable inserts include inserts made from fleece fabric and from hemp fabric. The insert 42 may be used with anyone of the caps 10, 22, 32 by stuffing the insert into the interior cavity of the cap to absorb moisture or urine excreted by the child. Alternatively, the cap 32 and insert 42 may incorporate a latching system. For example, the cap may include one or more Velcro® hooks and the insert itself may function as a loop and attaches to the one or more hooks. In an alternative embodiment, the inserts may be made from rice paper liners from Imse Vimse. Still alternatively, the inserts may be made from the same materials as currently available disposable diapers and from cotton or cotton-blend absorbent material.

In one exemplary embodiment, the caps 10, 22, 32 and the inserts 42 may include wetness indicator mechanism or technology to indicate when a child has urinated. Such technology is well known in the art and is described in U.S. Pat. No. 4,507,121 (Leung); U.S. Pat. No. 4,705,513 (Sheldon et al.); U.S. Pat. No. 5,354,289 (Mitchell et al.); and U.S. Pat. No. 5,690,624 (Sasaki et al.), their contents are expressly incorporated herein by reference as if set forth in full. Accordingly, the caps 10, 22, 32 and the inserts 42 described elsewhere herein may incorporate the wetness indicator aspect of the four described patents.

Although the preferred embodiments of the invention have been described with some specificity, the description and drawings set forth herein are not intended to be delimiting, and persons of ordinary skill in the art will understand that various modifications may be made to the embodiments discussed herein without departing from the scope of the invention, and all such changes and modifications are intended to be encompassed within the appended claims. Various changes to the changing cabs described elsewhere herein including changes to the texture, the appearance, the shape, the size, and outer housing or design indicia may be made. Accordingly, many alterations and modifications may be made by those having ordinary skill in the art without deviating from the spirit and scope of the invention.

What is claimed is:

1. A method for covering a child comprising:
removing a diaper; and
placing a cap comprising an exterior surface, an interior surface defining an interior cavity, and an opening having a largest measured opening dimension of between 1.5 inches to 4 inches over a urinary system of the child without covering a buttock of the child.

2. The method of claim 1, further comprising the step of leaving the child diaper-less to air out the child's bottom.

3. The method of claim 1, further comprising the step of weighing the child.

4. The method of claim 1, further comprising the step of taking pictures of the child.

5. The method of claim 1, further comprising the step of incorporating visual indicia to the exterior surface.

6. The method of claim 5, wherein the visual indicia comprises at least one of multiple colors, an animal design, a hat design, a car design, and a sport equipment design.

7. The method of claim 1, wherein the cap comprises an adjustable connector.

8. An apparatus for covering a child comprising:
a cap comprising an interior cavity and an elastic stretch band adjacent a perimeter defining an opening;
an absorbent insert positioned inside the interior cavity of the cap; and
wherein the opening is sized to fit around a genital area of the child.

9. The apparatus of claim 8, further comprising an adjustable connector.

10. The apparatus of claim 8, wherein the cap comprises an exterior surface and an interior surface defining an interior cavity.

11. The apparatus of claim 10, further comprising a visual indicia on the exterior surface.

12. The apparatus of claim 10, further comprising an adjustable connector and wherein the adjustable connector comprises at least one of a button and a button hole, male and female snap buttons, and VELCRO®.

13. The apparatus of claim 10, wherein the visual indicia comprises at least one of multiple colors, an animal design, a hat design, a car design, and a sport equipment design.

14. The apparatus of claim 8, wherein the absorbent insert comprises at least one of fleece fabric, hemp fabric, and rice paper liner.

15. An apparatus for covering a child comprising:
a cap comprising an exterior surface having an exterior color, an interior surface defining an interior cavity, and an elastic stretch band adjacent a perimeter defining an opening;
a decorative element adorns the exterior surface of the cap to provide a distinct visual indicia apart from the exterior color; and
wherein the opening is sized to contact a genital area of the child around a substantial portion of the opening.

16. The apparatus of claim 15, further comprising an adjustable connector.

17. The apparatus of claim 15, further comprising a visual indicia on the exterior surface.

18. The apparatus of claim 16, further comprising an adjustable connector and wherein the adjustable connector comprises at least one of a button and a button hole, male and female snap buttons, and VELCRO®.

19. The apparatus of claim 17, wherein the visual indicia comprises at least one of multiple colors, an animal design, a hat design, a car design, and a sport equipment design.

20. The apparatus of claim 15, further comprising an absorbent insert.

21. The apparatus of claim 20, wherein the absorbent insert comprises at least one of fleece fabric, hemp fabric, and rice paper liner.

22. The apparatus of claim 20, wherein the absorbent insert comprises a wetness indicator.

23. The apparatus of claim 20, wherein the absorbent insert is made from a cotton or a cotton-blend material.
